(19) United States
(12) Patent Application Publication
(10) Pub. No.: US 2009/0018405 A1
(43) Pub. Date: Jan. 15, 2009

(54) Exercise load measuring device

(75) Inventors: Toshihito Katsumura, Tokyo (JP); Ryotaro Kime, Tokyo (JP); Hikaru Suzuki, Kanagawa (JP); Yutaka Yokoyama, Kanagawa (JP); Katsumi Okazaki, Kanagawa (JP); Yukio Chitose, Kanagawa (JP); Ryu Watanabe, Kanagawa (JP)

Correspondence Address: Christopher J. Fildes;Fildes & Outland, P.C., Suite 2, 20916 Mack Avenue, Grosse Pointe Woods, MI, 48236, US

(73) Assignee: ASTEM Corporation

(21) Appl. No.: 12/002,345

(22) Filed: Dec. 17, 2007

(30) Foreign Application Priority Data
Jul. 13, 2007 (JP) JP2007-184397

(51) Int. Cl. A61B 5/024; A61B 5/1455

(52) U.S. Cl. 600/301

(57) ABSTRACT

There is provided an exercise load measuring device that can measure states of metabolism during exercise. The exercise load measuring device includes: a hemoglobin detecting portion 11 that includes a light emitting portion 111 that outputs near infrared lights having two wavelength, and two light receiving portions 112-1 and 112-2 that detect intensity of the near infrared lights and are placed at different distances from the light emitting portion, and detects hemoglobin concentration in muscle tissue of a subject; a heart rate detecting portion 12 that includes a light emitting portion 121 and a light receiving portion 122 and detects heart rate of the subject; and a main device 13, wherein the main device 13 includes an arithmetical operation portion 131 that arithmetically operates metabolic superiority, a display portion 132 that displays the metabolic superiority or the like, a storage portion 133 that stores arithmetical operation expressions, profile data of the subject, or the like, an input portion 134 that inputs data, and a warning portion 135 that issues a warning when a state where saccharometabolism is superior to lipid metabolism is continued for a certain time, and arithmetically operates metabolic superiority during exercise from oxygenated hemoglobin concentration, deoxygenated hemoglobin concentration, the heart rate, and weight and age of the subject.

The present application is based on and claims priority of Japanese patent application No. 2007-184397 filed on Jul. 13, 2007, the entire contents of which are hereby incorporated by reference.

### BACKGROUND OF THE INVENTION

1. Field of the Invention

The present invention relates to an exercise load measuring device that measures states of metabolism during exercise using heart rate, and oxygenated hemoglobin concentration and deoxygenated hemoglobin concentration in muscle tissue.

2. Description of the Related Art

A paper by Masatsugu Niwayama, Associate Professor at Shizuoka University reports that LEDs (light emitting diodes) having two wavelengths and two PDs (photodiodes) placed at different distances from the LEDs are provided, and oxygenated hemoglobin concentration and deoxygenated hemoglobin concentration in muscle tissue can be measured by intensity of light having passed through the muscle tissue, and an influence of a fat layer in a measurement spot that affects measurement accuracy can be corrected.

Masatsugu Niwayama, Associate Professor at Shizuoka University also logically and experimentally analyzes various causes of error in muscle oxygenation measurement, and suggests that a main cause of error in quantification is an influence of a fat layer thickness and scattering coefficient of the muscle tissue, the fat layer thickness and the scattering coefficient of the muscle have a significant influence on the absolute magnitude of hemoglobin concentration, while in the case of calculating oxygen saturation in the blood, the influence of the scattering coefficient of the muscle is reduced, and correction of the influence of the fat layer thickness can significantly improve quantitative characteristics, as causes of error in muscle oxygenation measurement using spatially resolved NIRS and correction thereof (for example, see “Causes of Error in Muscle Oxygenation Measurement Using Spatially Resolved NIRS and Correction of the Same” angiology 47-1, 17/20 (2007), Masatsugu Niwayama and four others).

### SUMMARY OF THE INVENTION

The present invention has an object to provide an exercise load measuring device that can measure a ratio between saccharometabolism and lipid metabolism, that is, metabolic superiority, an amount of lipid metabolism, and an exercise load during exercise.

During relatively light exercise for maintaining health, an exercise load measuring device that informs a subject of an exercise load uses a correlation between a ratio between oxygenated hemoglobin concentration and deoxygenated hemoglobin concentration in muscle tissue and respiratory quotient of expired gas, always observes metabolic superiority during exercise to detect an exercise load at which exercise can be continued for long hours and high lipid burning efficiency can be maintained, simultaneously measures heart rate indicating exercise intensity to detect an amount of lipid metabolism, and informs the subject of the exercise load and the amount of lipid metabolism.

The metabolic superiority is a ratio between energy required during exercise obtained from sugar in the blood and energy required during exercise obtained from lipid in the body, and a relationship between a lipid metabolism ratio obtained from the metabolic superiority, calorie consumption, and a lipid metabolism speed is expressed by Expression 1.

### [Expression 1]

Lipid metabolism speed=calorie consumption×lipid metabolism ratio (1)

According to the present invention, during relatively light exercise for maintaining health, the exercise load measuring device can use a correlation between oxygenated hemoglobin concentration in muscle tissue and respiratory quotient of expired gas, always observe metabolic superiority during exercise to detect an exercise load at which exercise can be continued for long hours and high lipid burning efficiency can be maintained, simultaneously measure heart rate indicating exercise intensity to detect an amount of lipid metabolism by exercise, and inform a subject of the exercise load and the amount of lipid metabolism.

### BRIEF DESCRIPTION OF THE DRAWINGS

FIG. 1 illustrates a configuration of an exercise load measuring device according to the present invention; and

FIG. 2 illustrates a surface shape of a main device of the exercise load measuring device according to the present invention.

### DETAILED DESCRIPTION OF THE PREFERRED EMBODIMENTS

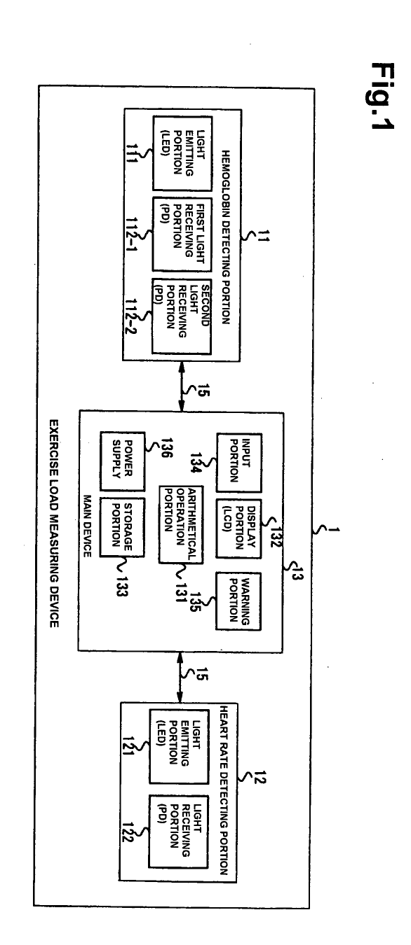

Now, a configuration of an exercise load measuring device according to the present invention will be described with reference to FIG. 1. The exercise load measuring device 1 includes a hemoglobin detecting portion 11 that optically detects hemoglobin concentration in muscle tissue, a heart rate detecting portion 12 that detects heart rate during exercise, and a main device 13 that uses the hemoglobin concentration, the heart rate, and fat layer thickness data to arithmetically operate and display an amount of lipid metabolism from the start of exercise and an exercise load. The hemoglobin detecting portion 11 and the main device 13, and the heart rate detecting portion 12 and the main device 13 are connected by cables 15.

The hemoglobin detecting portion 11 is a sensor that obtains optical data on the muscle tissue by an optical method required for arithmetically operating oxygenated hemoglobin concentration and deoxygenated hemoglobin concentration in the muscle tissue.

The hemoglobin detecting portion 11 is an optical measurement device that reads light absorption in muscle tissue required for arithmetically operating oxygenated hemoglobin concentration and deoxygenated hemoglobin concentration in muscle tissue of agonist muscle (thigh in this case) of a subject, and includes a light emitting portion 111 constituted by, for example, two LED (light emitting diode) elements that emit near infrared lights having two different wavelengths (for example, 770 nm and 830 nm) toward the agonist muscle, and a first light receiving portion 112-1 and a second light receiving portion 112-2 constituted by, for example, PDs (photodiodes) that are placed at different distances from the light emitting portion 111 and receive the near infrared lights from the light emitting portion 111. The lights having the wavelengths of 770 nm and 830 nm are applied in order from the light emitting portion 111 to a surface of a site with the agonist muscle (thigh) and pass through fat tissue and the muscle tissue to transmit data on the amounts of lights detected by the two light receiving portions 112-1 and 112-2 (the number of data: 4) to the main device 13. The amounts of lights detected by the light receiving portions 112-1 and 112-2 are different according to the distances between the light emitting portion 111 and the light receiving portions 112-1 and 112-2. Specifically, light having passed through only the fat tissue closer to the surface than the muscle tissue mainly reaches the first light receiving portion 112-1 placed close to the light emitting portion 111. Light having passed through both the fat tissue and the muscle tissue mainly reaches the second light receiving portion 112-2 remote from the light emitting portion 111. Light absorbing characteristics of the oxygenated hemoglobin and the deoxygenated hemoglobin in the muscle tissue differ with the wavelengths as parameters, and thus intensity of light for each wavelength received by each light receiving portion is detected, and the light intensity data is arithmetically operated using a method such as a spatially-resolved spectroscopy, thereby allowing the oxygenated hemoglobin concentration and the deoxygenated hemoglobin concentration in the muscle tissue to be detected. The oxygenated hemoglobin concentration and the deoxygenated hemoglobin concentration in the muscle tissue are indicative of states of exercise.

The heart rate detecting portion 12 is an optical measurement device that reads light absorption of artery required for arithmetically operating heart rate, and includes a light emitting portion 121 constituted by, for example, an LED that emits light having a wavelength well absorbed by the artery, and a light receiving portion 122 constituted by, for example, a PD placed to face the light emitting portion 121, and for example, the light emitting portion 121 and the light receiving portion 122 are placed to face each other at two tips of a clip in the form of a clothespin. For example, an amount of light detected by the light receiving portion 122 with an ear lobe held between the light emitting portion 121 and the light receiving portion 122 is changed by pulsation of blood flow. The heart rate detecting portion 12 transmits data (the number of data: 1) on the amount of light that is detected by the light receiving portion 122 and changed by pulsation of the blood to the main device 13.

The main device 13 is an arithmetical operation and display device that arithmetically operates the amount of lipid metabolism, the exercise load, the heart rate, and the metabolic superiority from data on the oxygenated hemoglobin concentration and the deoxygenated hemoglobin concentration obtained based on the light intensity data from the hemoglobin detecting portion 11, data on the heart rate obtained based on the light intensity data from the heart rate detecting portion 12, and fat layer thickness data of a hemoglobin detecting site and profile data (individual data) of the subject such as weight and age externally input, and displays the results on a display portion. The main device 13 includes an arithmetical operation portion 131, a display portion 132, a storage portion 133, an input portion 134, a warning portion 135, and a power supply 136. The main device 13 further includes a power switch, a data input key, and a set key.

The arithmetical operation portion 131 is means for arithmetically operating and obtaining the data on the oxygenated hemoglobin concentration and the deoxygenated hemoglobin concentration in the muscle tissue using the light intensity data from the hemoglobin detecting portion 11 and the fat layer thickness data, arithmetically operating and obtaining the heart rate using the light intensity data from the heart rate detecting portion 12, and arithmetically operating and obtaining the amount of lipid metabolism, the exercise load, the heart rate, and the metabolic superiority during exercise using the data on the oxygenated hemoglobin concentration and the deoxygenated hemoglobin concentration, the heart rate data, the individual data such as the weight and the age, or the like. The subject obtains the metabolic superiority during exercise to adjust the amount of exercise so as not to apply too heavy an exercise load for maintaining superiority of lipid metabolism, and continue the exercise. When a state where the exercise load becomes excessive and saccharometabolism is superior to lipid metabolism is continued for a certain time, the arithmetical operation portion 131 operates to issue a warning from the warning portion 135.

Calorie consumption is obtained from the heart rate data, and the weight data and the age data of the subject. The metabolic superiority and the lipid metabolism ratio are obtained from the oxygenated hemoglobin concentration and the deoxygenated hemoglobin concentration. Thus, the lipid metabolism speed expressed by Expression 1 can be obtained from the heart rate data, the weight data, the age data, the oxygenated hemoglobin concentration, and the deoxygenated hemoglobin concentration.

The display portion 132 has a display device constituted by a liquid crystal display device (LCD), and is means for displaying states of metabolism such as the amount of lipid metabolism, the exercise load, the heart rate, and the metabolic superiority that are arithmetical operation results, and individual profile data such as date, time, age, sex, fat layer thickness, and weight at the time of input of data.

The storage portion 133 is means for storing various arithmetical operation expressions used for the above described arithmetical operations, the input data, the arithmetical operation results, or the like.

The input portion 134 is means for inputting various data on the individual profile such as the fat layer thickness data, the age, the sex, the weight, or the like externally input.

The warning portion 135 is means for issuing a warning sound when the state where saccharometabolism is superior to lipid metabolism is continued for a certain time and informing the subject thereof, and is constituted by, for example, a speaker.

The power supply 136 is constituted by a primary battery such as a dry battery or a secondary battery, operates the main device 13, and supplies operation power to the hemoglobin detecting portion 11 and the heart rate detecting portion 12.

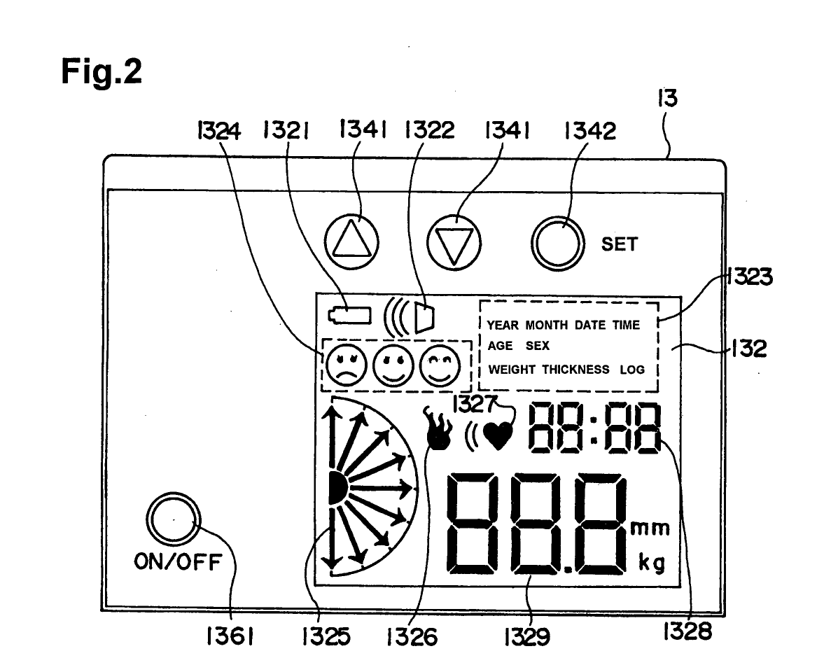

The shape of the surface of the main device 13 and an exemplary configuration of the display portion 132 will be described with reference to FIG. 2. On the surface of the main device 13, the display portion 132, a data input key 1341, a set key 1342, and a power switch 1361 are provided.

The display portion (LCD) 132 includes a battery remaining amount indication 1321 that indicates a remaining amount of the power supply battery, a warning volume indication 1322 that indicates the volume of warning, an input indication 1323 that indicates and selects functions in input of various data, an exercise load indication 1324 that indicates an exercise load that can be estimated from the amounts of oxygenated hemoglobin and deoxygenated hemoglobin, a metabolic superiority indication 1325 that indicates metabolic superiority by arrows, an exercise indication 1326 that indicates exercise being performed (measurement being performed), a pulse voice indication 1327 that indicates that a state of outputting pulses by voice is selected, a time indication 1328 that indicates date, time, exercise time, or the like, and a numerical value indication 1329 that indicates a numerical value in input of data and the amount of lipid metabolism, or the like. These indications are selectively set as required.

An aspect of use of the exercise load measuring device 1 according to the present invention will be described. First, the hemoglobin detecting portion 11 is brought into contact with the thigh (vastus lateralis muscle) and secured by a supporter or a bandage. Further, the main device 13 is fitted to a waist belt, the heart rate detecting portion 12 is fitted to the ear lobe, and the hemoglobin detecting portion 11 and the heart rate detecting portion 12 are connected to the main device 13 by the cables 15.

The power switch 1361 is turned on to turn the power on. Items required for data management and profiles (age, sex, weight) of the subject including a fat layer thickness are input by operating the data input key 1341. Then, the set button 1342 of the main device 13 is operated to obtain and arithmetically operate data on oxygenated hemoglobin concentration and deoxygenated hemoglobin concentration in muscle tissue and heart rate at rest for a certain time before the start of exercise, and record the data in the storage portion 133 of the main device 13. After a lapse of the certain time, the exercise indication 1326 is lit and 0.0 g is set to the numerical value indication 1329 to start measurement. When the subject starts the measurement, the metabolic superiority indication 1325 indicates metabolic superiority. The metabolic superiority indication 1325 indicates the metabolic superiority by nine stages, and the arrow indicating upward means superiority of lipid metabolism, and the arrow indicating downward means superiority of saccharometabolism.

During the exercise, the display portion 132 of the main device 13 always indicates the amount of lipid metabolism from the start of the exercise that is indicative of metabolic superiority and the exercise load.

The subject adjusts the exercise load so as not to be excessive for maintaining superiority of lipid metabolism, and continues the exercise. When the state where saccharometabolism is superior to lipid metabolism is continued for a certain time, an alarm of the main device 13 is sounded to give a warning to the subject.

Transmitting and receiving signals between the portions will be described. From the main device 13 to the hemoglobin detecting portion 11, a control signal and a power supply voltage are provided. From the hemoglobin detecting portion 11 to the main device 13, a voltage value (the number of data: 2 wavelengths×2 spots=4) is provided indicating the amount of light having transmitted through the fat tissue and the muscle tissue and detected by the two light receiving portions 112-1 and 112-2 among lights applied from the light emitting portion 111. From the main device 13 to the heart rate detecting portion 12, a control signal and a power supply voltage are provided. From the heart rate detecting portion 12 to the main device 13, a voltage value (the number of data: 1) is provided indicating the amount of light having transmitted through the ear lobe and detected by the light receiving portion 122 placed to face the light emitting portion 121 among lights applied from the light emitting portion 121.

The arithmetical operation portion 131 arithmetically operates the amount of lipid metabolism and the exercise load using data on the fat layer thickness, the weight and the age externally input, data on voltage values read by the light receiving portions 112-1 and 112-2 of the hemoglobin detecting portion and the light receiving portion 122 of the heart rate detecting portion 12, or the like, and outputs the arithmetical operation results on the display portion 132.

The principle of the arithmetical operation in the present invention will be described below. Generally, energy required for exercise is supplied by both saccharometabolism and lipid metabolism. At this time, in a state of exercise that is hard to continue for long hours, the saccharometabolism ratio is higher. On the other hand, in a state of exercise that can be continued for long hours, the lipid metabolism ratio is higher.

Though increasing the exercise load increases calorie consumption (the entire amount of metabolism), continuous exercise cannot be performed because of fatigue, thereby preventing much fat from burning. On the other hand, too low an exercise load causes superiority of lipid metabolism to increase efficiency, but calorie consumption itself is reduced, thereby preventing much fat from burning.

It is known that there is a point of a significant change from superiority of lipid metabolism to superiority of saccharometabolism in increasing exercise intensity at a certain rate. Exercise performed around the point most efficiently burns much fat.

The relationship between states of metabolism and expired gas will be described. The states of metabolism of a living body are calculated from the expired gas. A textbook of physiology “The Physiology of Training, 1st ed.” (Shukou Haga, Hideki Oono, Kyorin-Shoin Publishers, Jan. 20, 2003) defines the states of metabolism from a ratio between carbon dioxide produced and oxide consumed in expiration (respiratory quotient) expressed by Expression 2.

### [Expression 2]

Respiratory quotient=carbon dioxide produced/oxide consumed in expiration (2)

This respiratory quotient indicates the ratio between saccharometabolism and lipid metabolism, that is, metabolic superiority. An expired gas analysis reveals that the respiratory quotient is 0.85 at rest, 0.85 to 1.00 in superiority of saccharometabolism, and 0.71 to 0.85 in superiority of lipid metabolism.

It is experimentally known that the respiratory quotient has a strong correlation with the oxygenated hemoglobin concentration and the deoxygenated hemoglobin concentration.

Specifically, the respiratory quotient, that is, the metabolic superiority can be obtained from the relationship between the oxygenated hemoglobin concentration and the deoxygenated hemoglobin concentration.

For calculating the amount of lipid metabolism from energy consumption, the ratio between saccharometabolism and lipid metabolism is calculated from the respiratory quotient, and energy consumption corresponding to the lipid metabolism ratio is converted into an amount of fat, and thus an amount of fat burning can be calculated. Specifically, calorie consumption can be calculated from the weight and the age of the subject (a person during exercise) and the heart rate during exercise, the metabolic ratio of the energy consumption can be calculated from the ratio of hemoglobin concentration, and thus the amount of fat burning of the person during exercise can be calculated.

It is experimentally known that the ratio of lipid metabolism during aerobic exercise differs by sex, and thus sex difference can be used for calculating the amount of fat burning.

These arithmetical operation expressions are stored in the storage portion 133 of the main device 13, and the arithmetical operation is performed by the arithmetical operation portion 131.

In the embodiment, the data on the oxygenated hemoglobin concentration and the deoxygenated hemoglobin concentration is obtained using the intensity data of the light having passed through the muscle tissue obtained by the hemoglobin detecting portion 11 and the fat layer thickness data. However, the fat layer thickness data is used for reducing the influence of the presence of the fat layer and increasing measurement accuracy, and thus there is no need for using the fat layer thickness data if the measurement accuracy is within an allowable range, and it is only necessary that the arithmetical operation is performed using only the light intensity data detected by the hemoglobin detecting portion 11. In this case, the arithmetical operation portion 131 performs the arithmetical operation using only the light intensity data detected by the hemoglobin detecting portion 11.

The aspect of the present invention provides the exercise load measuring device 1 including the hemoglobin detecting portion 11 that detects hemoglobin in the blood of the subject, the heart rate detecting portion 12 that detects the heart rate of the subject, and the main device 13 that arithmetically operates the metabolic superiority during exercise using the oxygenated hemoglobin concentration and the deoxygenated hemoglobin concentration, wherein the hemoglobin detecting portion 11 includes the light emitting portion 111 that outputs the near infrared lights having two wavelengths, and the two light receiving portions 112-1 and 112-2 that detect the intensity of the near infrared lights having passed through the fat tissue and the muscle tissue of the subject and are placed at different distances from the light emitting portion 111, the heart rate detecting portion 12 includes the light emitting portion 121 that emits the near infrared light having a wavelength that passes through the blood, and the light receiving portion 122 that receives the near infrared light having passed through the blood, and the main device 13 obtains the oxygenated hemoglobin concentration and the deoxygenated hemoglobin concentration based on the data detected by the hemoglobin detecting portion 11 and the fat thickness, and arithmetically operates the metabolic superiority from the oxygenated hemoglobin concentration, the deoxygenated hemoglobin concentration, the heart rate, and the weight and the age of the subject.

Further, in the present invention, the light emitting portion 111 of the hemoglobin detecting portion 11 in the exercise load measuring device 1 is means for emitting the near infrared lights of 770 nm and 830 nm.

In the present invention, the main device 13 in the exercise load measuring device 1 includes the arithmetical operation portion 131 that arithmetically operates the metabolic superiority or the like, the display portion 132 that displays the metabolic superiority or the like, the storage portion 133 that stores the arithmetical operation expressions, the profile data of the subject, or the like, the input portion 134 that inputs the profile data of the subject, and the warning portion 135 that issues a warning when the state where saccharometabolism is superior to lipid metabolism is continued for a certain time.

The exercise load measuring device of the present invention includes: hemoglobin measuring means for measuring intensity data of light having passed through muscle tissue required for arithmetically operating oxygenated hemoglobin concentration and deoxygenated hemoglobin concentration in the muscle tissue of a subject; heart rate measuring means for obtaining data required for arithmetically operating heart rate of the subject; and a main device that includes input means of weight and age of the subject, arithmetically operates the oxygenated hemoglobin concentration and the deoxygenated hemoglobin concentration from the light intensity data, and arithmetically operates a load during exercise, metabolic superiority, a lipid metabolism speed, or an amount of lipid metabolism from the oxygenated hemoglobin concentration, the deoxygenated hemoglobin concentration, the heart rate, the weight, and the age.

In the present invention, the hemoglobin measuring means in the exercise load measuring device includes a light emitting portion that outputs near infrared lights having two wavelengths, and two light receiving portions that detect intensity of the near infrared lights having passed through fat tissue and the muscle tissue of the subject and are placed at different distances from the light emitting portion, the heart rate measuring means includes a light emitting portion that emits near infrared light having a wavelength that passes through the blood, and a light receiving portion that receives the near infrared light having passed through the blood, and the main device includes the input means of a thickness of a fat layer between the hemoglobin measuring means and the muscle tissue, obtains the oxygenated hemoglobin concentration and the deoxygenated hemoglobin concentration based on hemoglobin concentration data measured by the hemoglobin measuring means and the fat layer thickness, and arithmetically operates the lipid metabolism speed from the oxygenated hemoglobin concentration, the deoxygenated hemoglobin concentration, the heart rate, and the weight and the age of the subject.

Further, in the present invention, the main device in the exercise load measuring device includes an arithmetical operation portion that arithmetically operates the oxygenated hemoglobin concentration and the deoxygenated hemoglobin concentration based on the hemoglobin concentration data and the fat layer thickness, and arithmetically operates a ratio between saccharometabolism and lipid metabolism, that is, metabolic superiority, a display portion that displays the metabolic superiority or the like, a storage portion that stores arithmetical operation expressions, profile data of the subject, or the like, an input portion that inputs the profile data of the subject or the like, and a warning portion that issues a warning when a state where saccharometabolism is superior to lipid metabolism is continued for a certain time.

It is experimentally known that in the case of extremely light exercise, the light emitting portion 111 and the light receiving portion 112 of the hemoglobin detecting portion 11 can also function as the light emitting portion 121 and the light receiving portion 122 of the heart rate detecting portion 12. Thus, in the embodiment, the heart rate is detected by the heart rate detecting portion 12 that detects the heart rate, but the heart rate can be arithmetically operated using the light intensity data detected by the hemoglobin detecting portion 11. In this case, the hemoglobin detecting portion 11 or the heart rate detecting portion 12 has both functions.

In the above embodiment, optical heart rate detecting means that includes the light emitting portion 121 and the light receiving portion 122 that detect the heart rate at the ear lobe is used as the heart rate detecting portion 12. However, the heart rate detection means used as the heart rate detecting portion 12 includes various types of means such as wristwatch-type heart rate detection means that uses near infrared ray and always detects pulsation at a forearm, wristwatch-type means that measures heart rate only by placing a fingertip on a measurement portion, and means using a method of analyzing a bioelectric current generated in contraction of cardiac muscle with a chest belt applied to the chest. In the present invention, these heart rate detection means may be used in view of ease in handling or fitting.

In the above descriptions, the main device 13 is configured to be fitted to the waist belt of the subject, but in application of the present invention to a training machine such as a treadmill or an ergometer, the main device 13 can be incorporated into a monitor or the like of the training machine. Further, in this case, batteries and radio communication portions are provided in the hemoglobin detecting portion 11 and the heart rate detecting portion 12, and a radio communication portion is provided in the main device 13, thereby connecting the hemoglobin detecting portion 11 and the heart rate detecting portion 12 and the main device 13 by radio and eliminating the cables.

What is claimed is:

1. An exercise load measuring device comprising:
hemoglobin measuring means for measuring intensity data of light having passed through muscle tissue required for arithmetically operating oxygenated hemoglobin concentration and deoxygenated hemoglobin concentration in the muscle tissue of a subject;
heart rate measuring means for obtaining data required for arithmetically operating heart rate of the subject; and
a main device that includes input means of weight and age of the subject, arithmetically operates the oxygenated hemoglobin concentration and the deoxygenated hemoglobin concentration from the light intensity data, and arithmetically operates a load during exercise, metabolic superiority, a lipid metabolism speed, or an amount of lipid metabolism from the oxygenated hemoglobin concentration, the deoxygenated hemoglobin concentration, the heart rate, the weight, and the age.

2. The exercise load measuring device according to claim 1, wherein the hemoglobin measuring means includes a light emitting portion that outputs near infrared lights having two wavelengths, and two light receiving portions that detect intensity of the near infrared lights having passed through fat tissue and the muscle tissue of the subject and are placed at different distances from the light emitting portion,
the heart rate measuring means includes a light emitting portion that emits near infrared light having a wavelength that passes through the blood, and a light receiving portion that receives the near infrared light having passed through the blood, and
the main device includes the input means of a thickness of a fat layer between the hemoglobin measuring means and the muscle tissue, obtains the oxygenated hemoglobin concentration and the deoxygenated hemoglobin concentration based on hemoglobin concentration data measured by the hemoglobin measuring means and the fat layer thickness, and arithmetically operates the lipid metabolism speed from the oxygenated hemoglobin concentration, the deoxygenated hemoglobin concentration, the heart rate, and the weight and the age of the subject.

3. The exercise load measuring device according to claim 1, wherein the main device includes an arithmetical operation portion that arithmetically operates the oxygenated hemoglobin concentration and the deoxygenated hemoglobin concentration based on the hemoglobin concentration data and the fat layer thickness, and arithmetically operates a ratio between saccharometabolism and lipid metabolism, that is, metabolic superiority, a display portion that displays the metabolic superiority or the like, a storage portion that stores arithmetical operation expressions, profile data of the subject, or the like, an input portion that inputs the profile data of the subject or the like, and a warning portion that issues a warning when a state where saccharometabolism is superior to lipid metabolism is continued for a certain time.

4. The exercise load measuring device according to claim 1, wherein the hemoglobin measuring means includes a light emitting portion that outputs near infrared lights having two wavelengths, and two light receiving portions that detect intensity of the near infrared lights having passed through fat tissue and the muscle tissue of the subject and are placed at different distances from the light emitting portion, the heart rate measuring means includes a light emitting portion that emits near infrared light having a wavelength that passes through the blood, and a light receiving portion that receives the near infrared light having passed through the blood, and the main device includes an arithmetical operation portion that arithmetically operates the oxygenated hemoglobin concentration and the deoxygenated hemoglobin concentration based on the hemoglobin concentration data and the fat layer thickness, and arithmetically operates a ratio between saccharometabolism and lipid metabolism, that is, metabolic superiority, a display portion that displays the metabolic superiority or the like, a storage portion that stores arithmetical operation expressions, profile data of the subject, or the like, an input portion that inputs the profile data of the subject or the like, a warning portion that issues a warning when a state where saccharometabolism is superior to lipid metabolism is continued for a certain time, and input means of a thickness of a fat layer between the hemoglobin measuring means and the muscle tissue, the main device obtains the oxygenated hemoglobin concentration and the deoxygenated hemoglobin concentration based on hemoglobin concentration data measured by the hemoglobin measuring means and the fat layer thickness, and arithmetically operates the lipid metabolism speed from the oxygenated hemoglobin concentration, the deoxygenated hemoglobin concentration, the heart rate, and the weight and the age of the subject.
